(19) United States
(12) Patent Application Publication
(10) Pub. No.: US 2024/0404048 A1
(43) Pub. Date: Dec. 5, 2024

(54) TRAINING MEDICAL IMAGE ANNOTATION MODELS

(71) Applicant: KONINKLIJKE PHILIPS N.V., EINDHOVEN (NL)

(72) Inventors: Ye Li, Cambridge, MA (US); Jiahong Ouyang, Cambridge, MA (US); Li Chen, Cambridge, MA (US); Alvin Chen, Cambridge, MA (US)

(21) Appl. No.: 18/676,784

(22) Filed: May 29, 2024

Related U.S. Application Data
(60) Provisional application No. 63/469,579, filed on May 30, 2023.

(51) Int. Cl. G06T 7/00; G06T 7/11; G16H 30/40; G16H 50/20

(52) U.S. Cl. CPC G06T 7/0012; G06T 7/11; G16H 30/40; G16H 50/20; G06T 2207/10016; G06T 2207/10132; G06T 2207/20081

(57) ABSTRACT

Techniques for training models, using weakly-labeled data, to generate predictions based on medical images are disclosed. Models can be trained to perform feature localization, object detection, and/or segmentation. Weakly-labeled data can include unlabeled data or video-level labeled data. Medical imaging data is received including a first set comprising frame-level annotations and a second set comprising weakly-labeled data. A training dataset is generated comprising frame-level ground truth data, and a model is trained, using the training dataset, to generate predictions based on new medical imaging data. In some examples, the training data set may further include weakly-labeled data. In some examples, the training procedure uses a teacher model to generate frame-level localizations (pseudo-labels), which are used to train a student model whose weights can be adaptively transferred to the teacher model. Generated predictions can include frame-level feature localizations and/or video-level annotations.

### CROSS-REFERENCE TO RELATED APPLICATION

This application claims the benefit of and priority to U.S. Provisional Application No. 63/469,579 filed May 30, 2023, the contents of which are herein incorporated by reference.

### TECHNICAL FIELD

This application relates to feature detection and segmentation in medical images. More specifically, this application relates to training models to perform feature localization on medical images using weakly-labeled data.

### BACKGROUND

Various medical imaging modalities can be used for clinical analysis and medical intervention, as well as visual representation of the function of organs and tissues, such as magnetic resonance imaging (MRI), ultrasound (US), or computed tomography (CT). For example, lung ultrasound (LUS) is an imaging technique deployed at the point of care for the evaluation of pulmonary and infectious diseases, including COVID-19 pneumonia. Important clinical features—such as B-lines, merged B-lines, pleural line changes, consolidations, and pleural effusions—can be visualized under LUS, but accurately identifying these clinical features requires clinical expertise. Other imaging modalities and/or applications present similar challenges related to feature localization (e.g., detection and/or segmentation). Feature localization using artificial intelligence (AI)/machine learning (ML) models can aid in disease diagnosis, clinical decision-making, patient management, and the like. Other imaging modalities and/or applications can similarly benefit from automated feature detection and/or segmentation.

### SUMMARY

Apparatuses, systems, and methods for training medical image annotation models using weakly-labeled data are disclosed. For example, the disclosed techniques can be used to train one or more segmentation models and/or detection models to perform feature localization using medical imaging data, such as ultrasound imaging data. The disclosed techniques also include applying one or more trained models to perform feature localization.

In accordance with at least one example disclosed herein, a method of training a model is disclosed. A plurality of medical imaging data is received, including a first set of medical imaging data comprising frame-level annotations and a second set of medical imaging data comprising weakly-labeled data. A training dataset is generated, comprising frame-level ground truth data. The model is trained, using the generated training dataset, to generate predictions based on new medical imaging data, and the generated predictions include frame-level feature localizations.

In accordance with at least one example disclosed herein, a non-transitory computer-readable medium is disclosed carrying instructions that, when executed, cause a processor to perform operations. The operations include receiving a plurality of medical imaging data including a first set of medical imaging data comprising frame-level annotations and a second set of medical imaging data comprising weakly-labeled data, generating a training dataset comprising correlations between the frame-level annotations in the first set of medical imaging data and the weakly-labeled data in the second set of medical imaging data, and training a model using the generated training dataset to generate predictions based on new medical imaging data. The generated predictions include frame-level feature localizations.

In some implementations of the disclosed method and/or the non-transitory computer-readable medium, the weakly-labeled data comprises unlabeled data or video-level labeled data. In some implementations, the generated predictions include video-level annotations. In some implementations, the model is trained to determine a category for the new medical imaging data, and the category is selected from at least two categories. In some implementations, the video-level annotations are generated using a frame-to-video feature encoder. In some implementations, the new medical imaging data comprises an ultrasound video loop, and the plurality of medical imaging data comprises ultrasound videos, ultrasound frames, or both. In some implementations, the model is trained to generate a bounding box indicating a location of a target feature or delineate the location of the target feature. In some implementations, generating the training dataset includes pre-training a teacher model, using the first set of the medical imaging data comprising the frame-level annotations, to generate pseudo-labels, and training the model includes jointly training the teacher model and a student model using the second set of the medical imaging data comprising the weakly-labeled data, wherein the generated pseudo-labels are used as a ground truth for training the student model. In some implementations, the method and/or the operations further comprise transferring weights from the trained student model to the trained teacher model based on a transferring rate specified by an exponential moving average function. In some implementations, the transferring rate is adjusted based on evaluating performance of the student model using validation data. In some implementations, a frame included in the weakly-labeled data is weakly augmented for training of the teacher model and the frame is strongly augmented for training of the student model. In some implementations, the method and/or the operations further include evaluating quality of frame-level pseudo-labels included in the generated pseudo-labels based on video-level ground truth annotations or video-level pseudo-labels and filtering the frame-level pseudo-labels based on the quality. In some implementations, the method and/or the operations further include applying the trained model to the new medical imaging data to generate the predictions. In some implementations, the method and/or the operations further include evaluating an accuracy of the trained model using a testing dataset, and retraining the trained model using a different training dataset when the accuracy does not exceed a threshold accuracy. In some implementations, the model includes a baseline segmentation model or a baseline detection model.

Other examples disclosed herein include systems or apparatuses configured to perform one or more methods described herein, such as ultrasound imaging systems and/or computing systems.

### BRIEF DESCRIPTION OF THE DRAWINGS

FIG. 1 is a block diagram of an ultrasound imaging system arranged in accordance with principles of the present disclosure.

FIG. 2 is a block diagram illustrating a workflow for training detection and segmentation models using weakly-labeled data, according to principles of the present disclosure.

FIG. 3 is a block diagram illustrating a workflow for training a detection model using frame-level annotations, according to principles of the present disclosure.

FIG. 4 is a block diagram illustrating a workflow for training a segmentation model using frame-level annotations, according to principles of the present disclosure.

FIG. 5 is a block diagram illustrating stages of a process for training a model using weakly-labeled data, according to principles of the present disclosure.

FIG. 6A is a block diagram illustrating a workflow for training a model using video-level labeled data, according to principles of the present disclosure.

FIG. 6B is a block diagram illustrating a workflow for training a model using unlabeled data, according to principles of the present disclosure.

FIG. 7 is a flow diagram illustrating a process for training a model using weakly-labeled data, according to principles of the present disclosure.

### DESCRIPTION

The following description of certain examples is merely illustrative in nature and is in no way intended to limit the invention or its applications or uses. In the following detailed description of examples of the present apparatuses, systems and methods, reference is made to the accompanying drawings which form a part hereof, and in which are shown by way of illustration specific examples in which the described apparatuses, systems, and methods may be practiced. These examples are described in sufficient detail to enable those skilled in the art to practice the presently disclosed apparatuses, systems and methods, and it is to be understood that other examples may be utilized and that structural and logical changes may be made without departing from the spirit and scope of the present disclosure. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present technology is defined only by the appended claims.

Although artificial intelligence (AI) techniques, including machine learning (ML) techniques, have been applied to medical images, such as to generate annotations, various technical challenges arise. Existing technologies for training AI algorithms using medical images require extensive training data with expert human annotations (e.g., hundreds or thousands of frame-level annotations) of high clinical quality. For example, frame-by-frame annotation of a single cineloop ultrasound video can take hours to complete, which makes annotation at scale extremely challenging. As used herein, a medical image may refer to a visual representation of the interior of a body for clinical analysis and medical intervention. In an example, medical images can come from a variety of sources including X-rays, CT scans, MRI scans, ultrasound, and endoscopy. Similarly, although medical images often refer to the visual images themselves, as used herein medical images may refer to stored data that could be used to generate a visual image to represent the interior of a subject body.

Alternatively, video-level annotations could be used to provide supervision for training of ML models. Rather than manually and painstakingly annotating the location of features in every frame of an ultrasound video (e.g. via bounding boxes or segmentation masks), a single annotation could be provided for the entire video indicating presence or absence of one or more target features. This could be done with relative ease in a matter of seconds. However, training frame-level detection or segmentation models using video-level labels is technically challenging because the annotation information is incomplete, and existing systems have not adequately solved these challenges. That is, a single video-level tag does not contain the information about the location/size/shape of features in each ultrasound frame that existing technologies require to train an ML model to make such predictions. Similar challenges arise in relation to using unlabeled medical imaging data to train AI/ML models. Thus, standard methods based on known network architectures and ML training procedures cannot be applied.

The present disclosure describes systems and related methods for training models, using weakly-labeled data, to perform feature localization on medical imaging data, such as segmentation models or detection models. As used herein, “weakly-labeled data” can refer to medical imaging data that does not include frame-level annotations, such as medical imaging data that is unlabeled or medical imaging data that includes only video-level annotations, or medical imaging data that includes annotations for only a small number of frames (e.g., 5%, 10%, 15%). Weakly-labeled data may refer to medical imaging data that includes annotations for less than half of frames. Weakly-labeled data may refer to medical imaging data that includes annotations for less than a third of frames. Weakly-labeled data may refer to medical imaging data that includes annotations for less than ten percent of frames. Weakly-labeled data may refer to medical imaging data that includes annotations for less than one percent of frames. The disclosed technology includes a semi-supervised learning model design for ultrasound cineloop (e.g., video) feature localization (e.g., detection and segmentation) that utilizes both frame-level labeled and weakly-labeled data to improve prediction performance. The disclosed technology includes one or more models that can receive an ultrasound cineloop as an input, and the one or more models can generate frame-level feature localizations. In an example, localizations can include at least one of detection boxes, segmentation masks, or other indication of a location of a feature, and these localizations can be for each frame of the cineloop. In an example, the localization may be in conjunction with a display of the cineloop itself and one or more video-level predictions (e.g., a cineloop class).

The disclosed models can include a baseline detection or segmentation AI model trained with frame-level annotations. The baseline detection or segmentation AI model can be, for example, a deep learning network that takes individual frames from the ultrasound cineloop and produces bounding boxes or segmentation predictions corresponding to the locations of one or more target features in each frame. The baseline detection or segmentation AI model can be trained using supervised learning guided by frame-level annotation labels. That is, each image frame provided to the model during training is paired with a ground-truth annotation associated with that frame (e.g., bounding boxes in the case of detection, and free-form masks in the case of segmentation). The disclosed models can further include teacher models and student models trained using a semi-supervised teacher-student learning procedure that allows weakly-labeled cineloops to be used to supplement the training of the frame-level baseline AI model. For example, a teacher model can be trained to generate per-frame pseudo-labels for unlabeled images in a video clip (e.g., cineloop), and a student model can be trained to predict the pseudo-labels produced by the teacher. In some implementations, filtering can be applied, which may improve the accuracy of the pseudo-labels. In some implementations, training the student models and the teacher models can include applying adaptive learning. In these and other implementations, the teacher model and the student model are jointly trained in a semi-supervised process referred to as mutual learning, whereby the student and the gradually-progressing teacher are updated in a mutually beneficial manner. In some implementations, the weakly-labeled data used to train the teacher models and the student models includes video-level annotations, such as labels indicating a single binary class for a cineloop (e.g., indicating that the cineloop is either “positive” or “negative” for a target feature).

In some applications, the teacher-student training techniques disclosed herein may be flexible in allowing different types of data annotations to be combined in training. In some applications, video-level annotations may improve supervision of the training of frame-level detection and segmentation deep learning models. In some applications, the adaptive learning schemes may improve the robustness and consistency of the semi-supervised learning mechanism.

The technology disclosed herein may reduce or eliminate the need for time-consuming and expensive frame-by-frame annotation efforts in some applications. For example, in some applications, the disclosed technology may provide improved localization accuracy and robustness compared to existing baseline models, and may be more efficient in data and annotation usage.

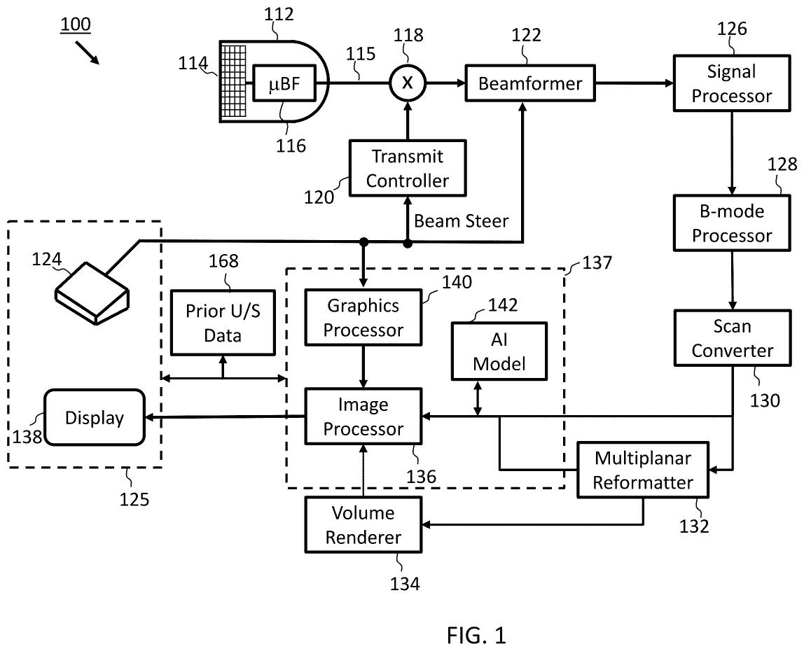

FIG. 1 is a block diagram of an ultrasound imaging system 100 arranged in accordance with principles of the present disclosure. In the ultrasound imaging system 100 of FIG. 1, an ultrasound probe 112 includes a transducer array 114 for transmitting ultrasonic waves and receiving echo information. The transducer array 114 may be implemented as a linear array, convex array, a phased array, and/or a combination thereof. The transducer array 114, for example, can include a two-dimensional array (as shown) of transducer elements capable of scanning in both elevation and azimuth dimensions for 2D and/or 3D imaging. The transducer array 114 may be coupled to a microbeamformer 116 in the probe 112, which controls transmission and reception of signals by the transducer elements in the array. In this example, the microbeamformer 116 is coupled by the probe cable to a transmit/receive (T/R) switch 118, which switches between transmission and reception and protects the main beamformer 122 from high-energy transmit signals. In some embodiments, the T/R switch 118 and other elements in the system can be included in the ultrasound probe 112 rather than in a separate ultrasound system base. In some embodiments, the ultrasound probe 112 may be coupled to the ultrasound imaging system via a wireless connection (e.g., WiFi, Bluetooth).

The transmission of ultrasonic beams from the transducer array 114 under control of the microbeamformer 116 is directed by the transmit controller 120 coupled to the T/R switch 118 and the beamformer 122, which receives input from the user's operation of the user interface (e.g., control panel, touch screen, console) 124. The user interface 124 may include soft and/or hard controls. One of the functions controlled by the transmit controller 120 is the direction in which beams are steered. Beams may be steered straight ahead from (orthogonal to) the transducer array, or at different angles for a wider field of view. The partially beamformed signals produced by the microbeamformer 116 are coupled via channels 115 to a main beamformer 122 where partially beamformed signals from individual patches of transducer elements are combined into a fully beamformed signal. In some embodiments, microbeamformer 116 is omitted and the transducer array 114 is coupled via channels 115 to the beamformer 122. In some embodiments, the system 100 may be configured (e.g., include a sufficient number of channels 115 and have a transmit/receive controller programmed to drive the array 114) to acquire ultrasound data responsive to a plane wave or diverging beams of ultrasound transmitted toward the subject. In some embodiments, the number of channels 115 from the ultrasound probe may be less than the number of transducer elements of the array 114 and the system may be operable to acquire ultrasound data packaged into a smaller number of channels than the number of transducer elements.

The beamformed signals are coupled to a signal processor 126. The signal processor 126 can process the received echo signals in various ways, such as bandpass filtering, decimation, I and Q component separation, and harmonic signal separation. The signal processor 126 may also perform additional signal enhancement such as speckle reduction, signal compounding, and noise elimination. The processed signals are coupled to a B-mode processor 128, which can employ amplitude detection for the imaging of structures in the body. The signals produced by the B-mode processor 128 are coupled to a scan converter 130 and a multiplanar reformatter 132. The scan converter 130 arranges the echo signals in the spatial relationship from which they were received in a desired image format. For instance, the scan converter 130 may arrange the echo signal into a two-dimensional (2D) sector-shaped format, or a pyramidal three-dimensional (3D) image. The multiplanar reformatter 132 can convert echoes, which are received from points in a common plane in a volumetric region of the body into an ultrasonic image of that plane, as described in U.S. Pat. No. 6,443,896 (Detmer).

A volume renderer 134 converts the echo signals of a 3D dataset into a projected 3D image as viewed from a given reference point, e.g., as described in U.S. Pat. No. 6,530,885 (Entrekin et al.) The 2D or 3D images may be coupled from the scan converter 130, multiplanar reformatter 132, and volume renderer 134 to at least one processor 137 for further image processing operations. For example, the at least one processor 137 may include an image processor 136 configured to perform further enhancement and/or buffering and temporary storage of image data for display on an image display 138. The display 138 may include a display device implemented using a variety of known display technologies, such as LCD, LED, OLED, or plasma display technology. The at least one processor 137 may include a graphics processor 140, which can generate graphic overlays for display with the ultrasound images. These graphic overlays can contain, e.g., standard identifying information such as patient name, date and time of the image, imaging parameters, and the like. For these purposes the graphics processor 140 receives input from the user interface 124, such as a typed patient name. The user interface 124 can also be coupled to the multiplanar reformatter 132 for selection and control of a display of multiple multiplanar reformatted (MPR) images. The user interface 124 may include one or more mechanical controls, such as buttons, dials, a trackball, a physical keyboard, and others, which may also be referred to herein as hard controls. Alternatively or additionally, the user interface 124 may include one or more soft controls, such as buttons, menus, soft keyboard, and other user interface control elements implemented for example using touch-sensitive technology (e.g., resistive, capacitive, or optical touch screens). One or more of the user controls may be co-located on a control panel. For example one or more of the mechanical controls may be provided on a console and/or one or more soft controls may be co-located on a touch screen, which may be attached to or integral with the console.

The at least one processor 137 may also perform the functions associated with training models using weakly-labeled data, as described herein. For example, the processor 137 may include or be operatively coupled to an AI model 142. The AI model 142 can include various models, such as a baseline detection or segmentation AI model trained using frame-level annotations and/or one or more teacher models and student models trained using a semi-supervised procedure, as described herein. The AI model 142 can be trained using weakly-labeled data. The AI model 142 can comprise one or more detection models, segmentation models, or combinations thereof.

A “model,” as used herein, can refer to a construct that is trained using training data to make predictions or provide probabilities for new data items, whether or not the new data items were included in the training data. For example, training data for supervised learning can include items with various parameters and an assigned classification. A new data item can have parameters that a model can use to assign a classification to the new data item. As another example, a model can be a probability distribution resulting from the analysis of training data, such as a likelihood of an n-gram occurring in a given language based on an analysis of a large corpus from that language. Examples of models include, without limitation: AI models, ML models, neural networks, support vector machines, decision trees, Parzen windows, Bayes, clustering, reinforcement learning, probability distributions, decision trees, decision tree forests, and others. Models can be configured for various situations, data types, sources, and output formats.

In some implementations, the AI model 142 can include a neural network with one or multiple input nodes that receive training datasets. The input nodes can correspond to functions that receive the input and produce results. These results can be provided to one or more levels of intermediate nodes that each produce further results based on a combination of lower-level node results. A weighting factor can be applied to the output of each node before the result is passed to the next layer node. At a final layer, (“the output layer,”) one or more nodes can produce a value classifying the input that, once the model is trained, can be used to generate predictions based on medical imaging data (e.g., to perform detection tasks or segmentation tasks), and so forth. In some implementations, such as deep neural networks, a model can have multiple layers of intermediate nodes with different configurations, can be a combination of models that receive different parts of the input and/or input from other parts of the deep neural network, or are convolutions-partially using output from previous iterations of applying the model as further input to produce results for the current input. In some implementations, the AI model 142 can include one or more convolutional neural networks.

A model can be trained with supervised learning. Testing data can then be provided to the model to evaluate accuracy of the trained model and/or validate the trained model. Testing data can be, for example, a portion of the training data (e.g., 10%) held back to use for evaluation of the model. To evaluate accuracy, output from the model can be compared to the desired and/or expected output for the training data and, based on the comparison, the model can be modified, such as by changing weights between nodes of a neural network and/or parameters of the functions used at each node in the neural network (e.g., applying a loss function). Based on the results of the model evaluation, and after applying the described modifications, the model can then be retrained to evaluate new medical imaging data.

Although described as separate processors, it will be understood that the functionality of any of the processors described herein (e.g., processors 136, 140, 142) may be implemented in a single processor (e.g., a CPU or GPU implementing the functionality of processor 137) or fewer number of processors than described in this example. In yet other examples, the AI model 142 may be hardware-based (e.g., include multiple layers of interconnected nodes implemented in hardware) and be communicatively connected to the processor 137 to output to processor 137 the requisite image data for generating ultrasound images. While in the illustrated embodiment, the AI model 142 is implemented in parallel and/or conjunction with the image processor 136, in some embodiments, the AI model 142 may be implemented at other processing stages, e.g., prior to the processing performed by the image processor 136, volume renderer 134, multiplanar reformatter 132, and/or scan converter 130. In some embodiments, the AI model 142 may be implemented to process ultrasound data in the channel domain, beamspace domain (e.g., before or after beamformer 122), the IQ domain (e.g., before, after, or in conjunction with signal processor 126), and/or the k-space domain. As described, in some embodiments, functionality of two or more of the processing components (e.g., beamformer 122, signal processor 126, B-mode processor 128, scan converter 130, multiplanar reformatter 132, volume renderer 134, processor 137, image processor 136, graphics processor 140, etc.) may be combined into a single processing unit or divided between multiple processing units. The processing units may be implemented in software, hardware, or a combination thereof. For example, AI model 142 may include one or more graphical processing units (GPU). In another example, beamformer 122 may include an application specific integrated circuit (ASIC).

The at least one processor 137 can be coupled to one or more computer-readable media (not shown) included in the system 100, which can be non-transitory. The one or more computer-readable media can carry instructions and/or a computer program that, when executed, cause the at least one processor 137 to perform operations described herein. A computer program may be stored/distributed on any suitable non-transitory medium, such as an optical storage medium or a solid-state medium supplied together with or as part of other hardware, but may also be distributed in other forms, such as via the Internet or other wired or wireless telecommunication systems. Furthermore, the different embodiments can take the form of a computer program product accessible from a computer-readable medium providing program code for use by or in connection with a computer or any device or system that executes instructions. For the purposes of this disclosure, a computer-readable medium can generally be any tangible apparatus that can contain, store, communicate, propagate, or transport the program for use by or in connection with the instruction execution device. The computer-readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and/or an optical disk. Optical disks may include compact disk read only memory (CD-ROM), compact disk-read/write (CD-R/W), and/or DVD.

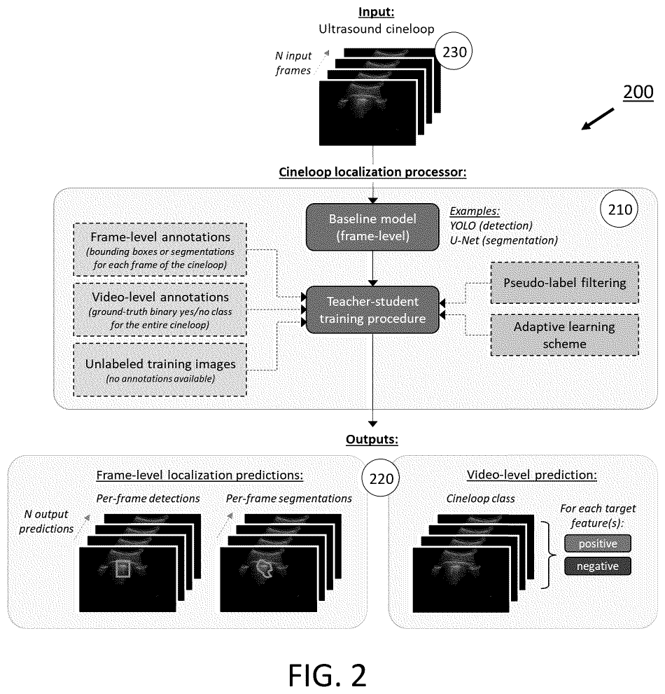

FIG. 2 is a block diagram illustrating a workflow 200 for training detection and segmentation models using weakly-labeled data, according to principles of the present disclosure. For example, the workflow 200 can be used to train one or more models included in the AI model 142 of FIG. 1. The workflow 200 includes applying a cineloop localization processor 210 to generate outputs 220 comprising frame-level localization predictions (e.g., detections and/or segmentations) and video-level predictions (e.g., cineloop class), based on inputs 230 comprising one or more ultrasound cineloops, each cineloop including N input frames. The cineloop localization processor 210 includes a baseline model, which can be a baseline detection or segmentation AI model trained with frame-level annotations, and a teacher-student training procedure for training teacher models and student models using weakly-labeled data.

The cineloop localization processor 210 receives the inputs 230 comprising the one or more ultrasound cineloops (e.g., videos). The inputs 230 can be acquired for example, using the system 100 of FIG. 1. The baseline model of the cineloop localization processor 210 processes the inputs 230 by taking individual frames from the ultrasound cineloop and producing bounding boxes or segmentation predictions corresponding to the locations of one or more target features in each frame. The baseline model can be trained using supervised learning guided by frame-level annotation labels. In some implementations, the baseline model can be a deep learning network. Additionally or alternatively, the baseline model can use the YOLO (You Only Look Once) architecture (e.g., for detection) and/or the U-Net architecture (e.g., for segmentation).

The teacher-student training procedure of the cineloop localization processor 210 can be a semi-supervised teacher-student learning procedure that allows unlabeled cineloops included in the inputs 230 to be used to supplement the training of the baseline model of the cineloop localization processor 210. In the procedure, a teacher model learns to generate per-frame pseudo-labels from unlabeled images in one or more ultrasound cineloops, and a student model learns to predict the pseudo-labels produced by the teacher. Additionally or alternatively, the teacher-student training procedure can use video-level annotations for the one or more ultrasound cineloops, such as video-level labels indicating a binary class (e.g., whether the cineloop is “positive” or “negative” for a target class). The teacher model and the student model are jointly trained in a semi-supervised process referred to as mutual learning, whereby the student and the gradually progressing teacher are updated in a mutually beneficial manner. In some implementations, the teacher-student learning procedure includes applying pseudo-label filtering to improve accuracy of pseudo-labels generated using the teacher model. In some implementations, the teacher-student learning procedure includes applying an adaptive learning scheme, which can gradually transfer weights from the student model to the teacher model (or from the teacher model to the student model), instead of using common backpropagation techniques.

The cineloop localization processor 210 provides the outputs 220 using the baseline model, the teacher model, and/or the student model. The outputs 220 can include frame-level localization predictions, such as per-frame detections or per-frame segmentations, and the outputs 220 can include a display of frame-level feature localizations (e.g., detection boxes or segmentation masks for each frame of the cineloop in conjunction with a display of the cineloop itself). Additionally or alternatively, the outputs 220 can include video-level predictions, and the outputs 220 can include display of the video-level predictions (e.g., the cineloop class).

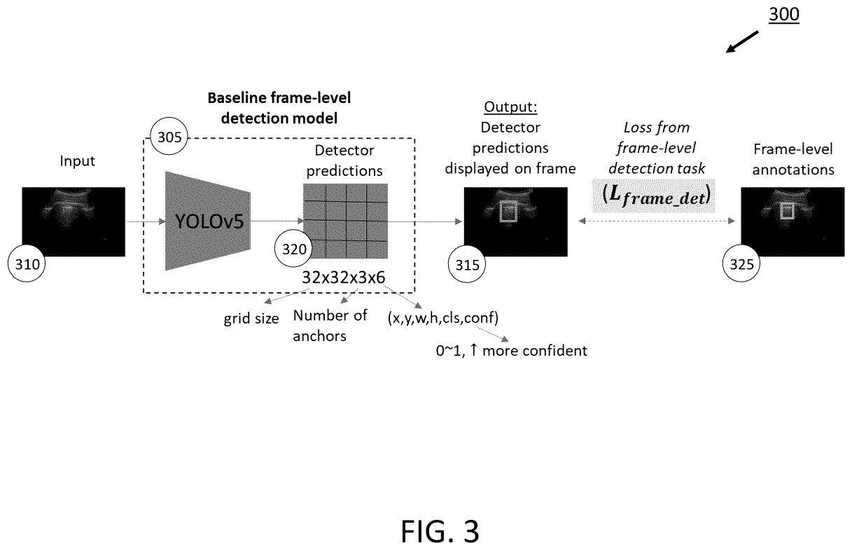

FIG. 3 is a block diagram illustrating a workflow 300 for training a detection model 305 using frame-level annotations, according to principles of the present disclosure. When trained using the workflow 300, the detection model 305 can be applied to detect one or more target features in frames of medical imaging data, such as by generating bounding boxes 315. The detection model 305 can be, for example, the baseline model of the cineloop localization processor 210 of FIG. 2. Additionally or alternatively, the detection model 305 can be included in the AI model 142 of FIG. 1. In the illustrated embodiment, the detection model 305 is based, at least in part, on the YOLO (You Only Look Once) architecture. In some implementations, other and/or additional architectures are used. The detection model 305 receives one or more frames as an input 310 and generates detection candidates 320 (e.g., indications of likely target features, such as bounding boxes) in the form of 6-dimensional vectors containing the following elements: x coordinate, y coordinate, width, height, classification probability, and confidence of the prediction (within the value range of 0 to 1). A plurality of such detection candidates 320 can be generated for each frame (e.g., as many as 32*32*3 detections may be generated per frame). Predicted bounding boxes 315 are then compared with ground truth annotations on the frame to regularize predictions in shape (L2 loss), classification class (cross entropy loss) and/or confidence (amount of overlap with frame-level annotation, L2 loss). The predicted bounding boxes 315 and/or the ground truth annotations 325 can be displayed on the frame. A loss, L_{frame_det}, from the frame-level detection task can be determined by comparing the predicted boxes and the ground truth annotations. The losses are back-propagated to adjust the weights of the model to improve the predictions in the next iteration. In some embodiments, the workflow 300 may be performed until the loss reaches a minimum and/or acceptable value.

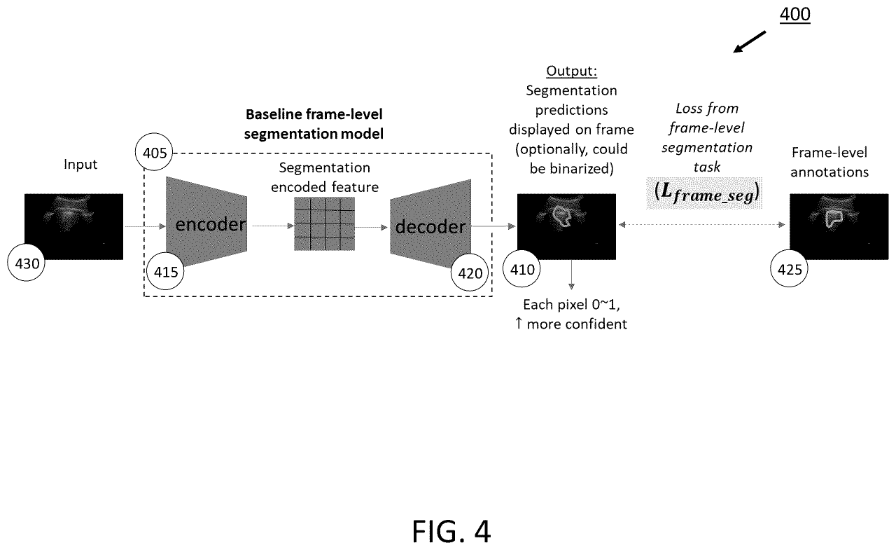

FIG. 4 is a block diagram illustrating a workflow 400 for training a segmentation model 405 using frame-level annotations, according to principles of the present disclosure. When trained using the workflow 400, the segmentation model 405 can be applied to detect one or more target features in frames of medical imaging data, such as by generating segmentation predictions 410 comprising boundaries of one or more target features. The segmentation model 405 can be, for example, the baseline model of the cineloop localization processor 210 of FIG. 2. Additionally or alternatively, the segmentation model 405 can be included in the AI model 142 of FIG. 1. In the illustrated embodiment, the segmentation model 405 is based, at least in part, on the U-Net architecture, which may use an encoder 415 and a decoder 420 arranged in an encoder-decoder architecture to output a segmentation prediction 410 comprising a segmentation map of the same width and height dimensions as the input frames. In some implementations, other and/or additional architectures are used. Each pixel in the segmentation map includes a confidence score between 0 and 1. During training, the segmentation map is compared with frame-level segmentation annotations 425 to calculate the segmentation loss. During inference, the predicted probability map can be binarized (for example, using thresholding) to serve as the frame-level output. In the workflow 400, the segmentation model 405 receives one or more frames of medical imaging data as an input 430 and generates segmentation predictions 410 (e.g., segmentation maps for the one or more frames) based on the input 430. In some implementations, the segmentation predictions 410 can be displayed on the one or more frames. A loss, L_{frame_seg}, from the frame-level segmentation task can be determined by comparing the segmentation predictions 410 to frame-level annotations 425 for the frames. The losses are back-propagated to adjust the weights of the model to improve the predictions in the next iteration. In some embodiments, the workflow 400 may be performed until the loss reaches a minimum and/or acceptable value.

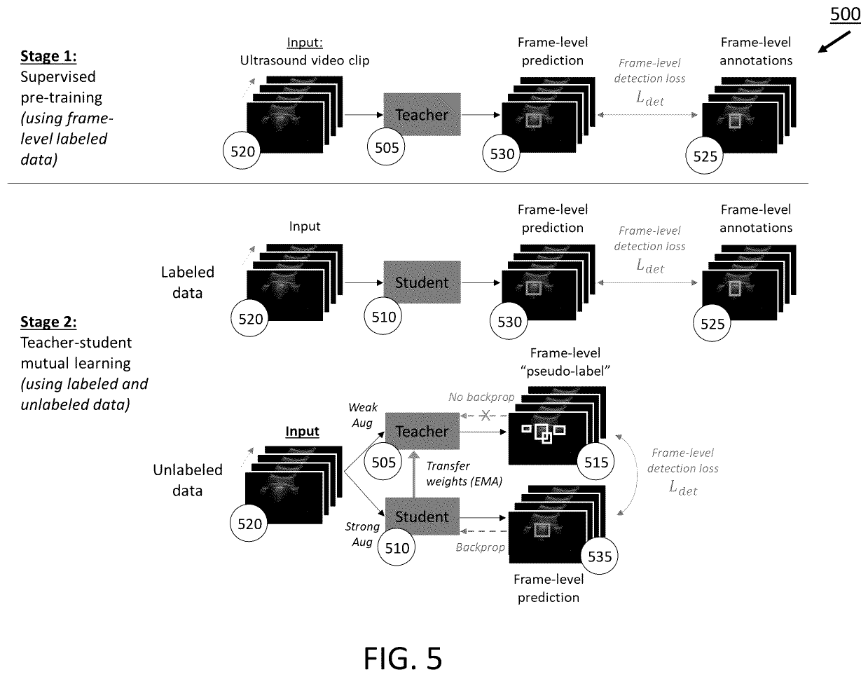

FIG. 5 is a block diagram illustrating stages of a process 500 for training a model using weakly-labeled data, according to principles of the present disclosure. When trained using the process 500, the model can be used to generate predictions (e.g., frame-level predictions and/or video-level predictions) based on medical imaging data (e.g., ultrasound cineloops). The process 500 can be performed, for example, by the cineloop localization processor 210 of FIG. 2. Additionally or alternatively, the model trained using the process 500 can be included in the AI model 142 of FIG. 1. The process 500 includes a Stage 1 (i.e., first stage) and a Stage 2 (i.e., second stage). Using the process 500, for each frame of a video clip (e.g., cineloop), a teacher model 505 is trained to generate a frame-level pseudo-label 515 on a weakly-augmented version of the input 520 (e.g., an image). As used herein, augmenting the input can include modifying one or more characteristics of an image, such as an amount of scaling, rotation, or the like. Accordingly, a weakly-augmented image can be an image with a relatively small amount of augmentation, as compared to a different image, such as an image that is scaled or rotated by a small percentage (e.g., 1%, 5%, 10%, 20%, 30%). A student model 510 then uses the generated pseudo-label 515 as the ground-truth for its own input, which is a strongly-augmented version of the same input 520.

At Stage 1 of the process 500, the teacher model 505 receives an input 520 comprising an ultrasound video clip. A training dataset is generated using the input 520, and the teacher model 505 is pre-trained for a set number of epochs using a portion of the training dataset that includes frame-level annotations 525 (e.g., bounding boxes and/or segmentations). The pre-training of the teacher model 505 can use supervised learning, such as training as described in the workflow 300 of FIG. 3 or the workflow 400 of FIG. 4. This initial supervised pre-training stage allows the teacher model 505 to be sufficiently optimized to produce reasonable frame-level predictions 530 based on received medical imaging data, which can be used as pseudo-labels.

At Stage 2 of the process 500, the pre-trained teacher model 505 is relied upon to generate pseudo-labels 515 to train the student model 510. Meanwhile, the student model 510 is initialized as a copy of the pre-trained teacher model 505 (e.g., using the same network structure and weights). The two models are jointly trained in a process referred to as mutual learning, such that the student model 510 and the gradually progressing teacher model 505 are updated in a mutually beneficial manner.

To perform mutual learning at Stage 2 of the process 500, an input 520 is received, which can be an unlabeled medical image. The input 520 is weakly augmented and provided to the teacher model 505, and the same input 520 is strongly augmented and provided to the student model 510. The teacher model 505 generates a prediction using the weakly-augmented image to be used as a pseudo-label 515, and the student model 510 generates a frame-level prediction 535 using the strongly augmented image. The frame-level prediction 535 can be a set of bounding boxes (e.g., when the models are trained to perform detection) or free-form masks (e.g., when models are trained to perform segmentation). Because the student model 510 receives the strongly augmented image, the student model's 510 prediction task is more challenging and error-prone. To be successful, the student model 510 is required to learn augmentation-agnostic representations of the underlying features in the image.

By contrast, predictions generated by the teacher model 505 are likely to have greater accuracy, and these predictions are used as pseudo-labels 515. In the absence of true frame-level ground-truth (e.g., user-generated annotations for frames), the teacher-generated pseudo-labels 515 serve as the ground-truth against which the student's predictions 535 are compared. In other words, pseudo-labeled images provided by the teacher model 505 can be used to generate a training dataset that is used to train the student model 510 when no ground-truth data is available for training, thus allowing the models to be trained using unlabeled data. A loss is computed by evaluating the difference between the student predictions 535 and the teacher pseudo-labels 515. The loss is back propagated through the student network in order to update the student model's 510 weights.

The training of the student model 510 and the teacher model 505 continues for a number of epochs, and at the end of each epoch, the updated weights of the student model 510 are transferred to the teacher model 505 via an exponential moving average (EMA) process. Note that, unlike for the student model 510, backpropagation is not applied to the teacher model 505. The EMA updates allow the teacher model 505 to be refined at a slower rate (but with better stability) compared to the student model 510. The process 500 prioritizes stability of the teacher model 505 to prevent the teacher-generated pseudo-labels 515 from becoming an unreliable ground-truth, which in turn could cause the entire mutual learning process to degrade. The EMA process to transfer the weights of the student model 510 to the teacher model 505 can use a weighted sum of the student model 510 weights and the teacher model 505 weights before the update, which can be expressed as:

$${θ}_{teacher} ← α \cdot {θ}_{teacher} + ( 1 - α ) \cdot {θ}_{student}$$

Here, a (referred to as the “keep rate”) is an important hyperparameter that controls how quickly the student model 510 transfers its newly learned knowledge (e.g., weights) to the teacher model 505. A large EMA keep rate (a approaching 1) means being conservative as the student model 510 is allowed to only migrate very little of the newly learned knowledge back to the teacher model 505; this will result in a robust but slow learning teacher model 505, which can take many epochs to train or not train at all. By contrast, a small EMA keep rate (a closer to 0) means being aggressive, which allows the student model 510 to migrate a lot of the newly learned knowledge back to the teacher model 505 in each epoch. However, some of the newly learned knowledge may not be correct in the sense they do not help increase validation performance on unseen data. As a result, this can lead to a fast but unstable learning. In some implementations, rather than applying a constant keep rate a, an adaptive learning scheme can be applied using an updating function to adaptively determine a keep rate based on the true performance of the teacher model 505 and student model 510 during training, as measured by their respective performance on unseen validation data. The adaptive EMA updating function prevents the student model 510 from transferring too much knowledge to the teacher model 505 (transfer weights more slowly) when its performance is bad and promotes the student model 510 to migrate more knowledge to the teacher model 505 (transfer weights more quickly) when its performance is much better than the teacher model 505. Accordingly, the keep rate a can be adjusted based on performance of the student model 510. The adaptive EMA updating function can be tuned depending on data and/or particular use cases. For example, the adaptive EMA updating function can be determined empirically, and student and teacher performance can be monitored during training (e.g., during each of a plurality of epochs) to select a preferred or acceptable EMA keep rate.

In some embodiments, the student model 510 can instead be adaptively modified based on performance of the teacher model 505 using an inverse adaptive EMA learning scheme. The inverse adaptive EMA learning scheme can help to boost the student by transferring the teacher's weights back to the student when the student has a worse performance than the teacher, such as when the student is presented with a series of particularly challenging images during the training process. In some implementations, the inverse adaptive EMA learning scheme can use an inverse adaptive EMA updating function, which can be tuned depending on data and/or particular use cases. For example, the inverse adaptive EMA updating function can be determined empirically, and student and teacher performance can be monitored during training (e.g., during each of a plurality of epochs) to select a preferred or acceptable EMA keep rate.

Because the teacher model 505 is more stable, as compared to the student model 510, the teacher model 505 is typically used as the final model for deployment. For example, the teacher model 505 may be deployed as at least part of AI model 142.

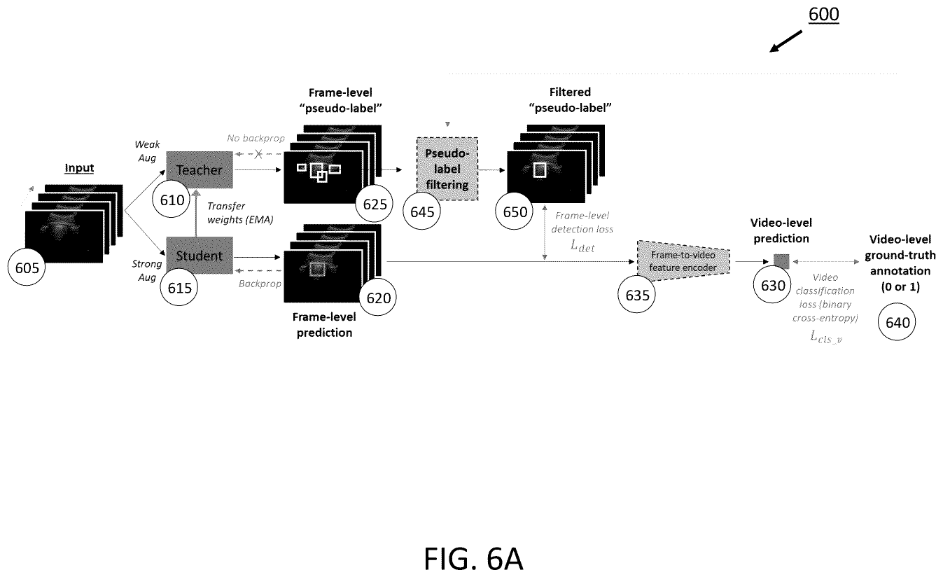

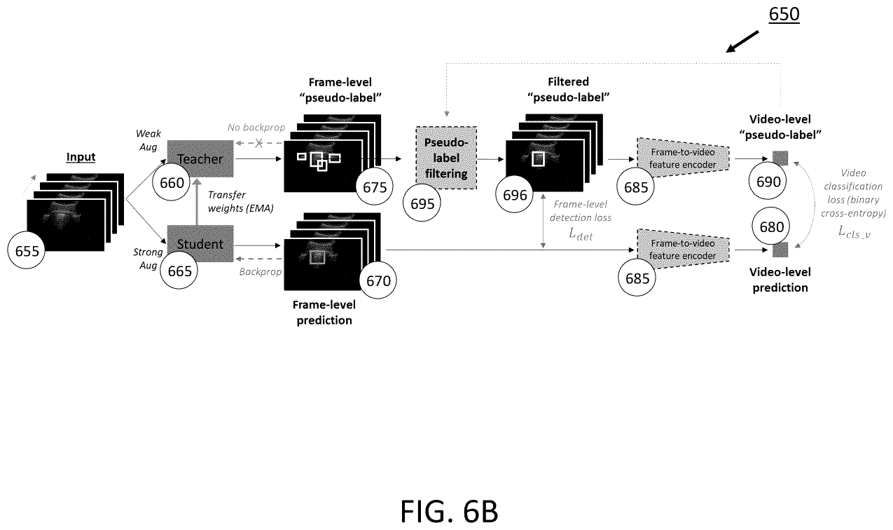

FIG. 6A is a block diagram illustrating a workflow 600 for training a model using video-level labeled data, according to principles of the present disclosure. When trained using the workflow 600, the model can be applied to generate predictions based on medical imaging data, such as frame-level predictions and/or video-level predictions. The workflow 600 can be performed, for example, by the cineloop localization processor 210 of FIG. 2. Additionally or alternatively, the model trained using the workflow 600 can be included in the AI model 142 of FIG. 1. Video-level labels included in the video-level labeled data can include a single binary class for a cineloop, such as an indication of whether the cineloop is positive or negative for one or more target features. Additionally or alternatively, video-level labels may consist of multiple categories (e.g., “normal”, “mild”, “moderate”, “severe”), a numerical score (e.g., severity score 0, 1, 2, or 3), or the like.

To train the model, an input 605 is received comprising medical imaging data, such as ultrasound cineloops comprising a plurality of frames. The received medical imaging data includes at least some video-level annotations, such as category labels. The workflow 600 can include applying teacher-student training using the video-level annotations, such as by pre-training a teacher model 610, as described in Stage 1 of the process 500 of FIG. 5, and applying mutual learning using the pre-trained teacher model 610 and a student model 615, as described in Stage 2 of the process 500 of FIG. 5. For example, the teacher model 610 can be pre-trained using a portion of the input 605 that includes frame-level annotations, and the teacher model 610 and the student model 615 can be jointly trained using a portion of the input 605 that includes video-level annotations. The teacher model 610 and the student model 615 are both trained to generate frame-level predictions (e.g., predictions 620 generated by the student model 615), and the frame-level annotations generated by the teacher model are used as pseudo-labels 625 to serve as a ground truth for training the student model 615.

To enable video-level supervision, per-frame predictions are aggregated into a one or more video-level predictions 630. This can be achieved using a frame-to-video feature encoder 635, which combines predictions from a plurality of frames into a video-level prediction 630. Generated video-level predictions 630 can be compared against video-level ground truth annotations 640 to determine a loss, L_{cls_v} for the video-level predictions 630. The loss L_{cls_v} is back propagated to improve accuracy of video-level predictions 630.

The frame-to-video feature encoder 635 processes frame-level predictions (e.g., 620) from the baseline detection/segmentation network and outputs a video-level classification prediction 630 for the video input (e.g., included in 605). The video-level prediction 630 is then compared against the true video-level ground-truth annotation 640 for that clip (in the absence of frame-level ground-truth annotations). The video-level loss is computed as a function of the difference between prediction and ground truth; this occurs during the mutual learning stage of the teacher-student training. The video-level classification loss can be a binary cross-entropy loss. During training, this loss is back propagated through the student network to update the student's weights.

In some implementations, the workflow 600 includes applying a filtering component 645 to improve accuracy of pseudo-labels 625 generated by the teacher model 610. The effectiveness of the mutual learning training process may be at least partially dependent on the quality of pseudo-labels 625 generated by the teacher model 610, since the pseudo-labels 610 serve as the ground-truth for the student model 615. In some implementations, lower quality pseudo-labels 625 can be filtered away using a filtering algorithm, so that the student model 615 is trained only using filtered pseudo-labels 650, which have been filtered using the filtering component 645 to include only generated pseudo-labels 625 determined to have a quality beyond a threshold level.

To evaluate quality of pseudo-labels 625, a filtering algorithm of the filtering component 645 receives an input comprising a batch of frame-level pseudo-labels 625 predicted by the teacher model 610. If a video-level ground truth annotation (e.g., 640) is available, the filtering algorithm compares each frame-level pseudo-label 625 to a corresponding video-level ground truth annotation. If no video-level ground truth annotation is available, the filtering algorithm compares each frame-level pseudo-label 625 to a corresponding video-level pseudo-label (e.g., generated by the teacher model 610). If the video-level annotation (or video-level pseudo-label) is negative (suggesting that the target feature is not present in the video), all frame-level pseudo-labels 625 for that feature are removed. On the other hand, if the video-level annotation (or video-level pseudo-label) is positive for the target feature, the algorithm checks whether there is at least one frame-level pseudo-label present (above a pre-defined threshold t1). In the case where there is at least one frame-level pseudo-label present, the algorithm removes the frame-level pseudo-labels 625 if their maximum confidence scores are below another pre-defined threshold t2. If none of the maximum confidence scores of the predicted frame-level pseudo-labels 625 are above the pre-defined threshold t1, the predicted frame-level pseudo-label 625 that has the maximum confidence score is kept and used as the final predicted frame-level pseudo-label 625 for the entire video clip. Based on the foregoing filtering operations, the filtering component 645 outputs filtered pseudo-labels 650, which can be used to train the student model 615 and/or provided to the frame-to-video feature encoder 635 to generate video-level predictions 630. Other variations may be applied to evaluate quality of pseudo-labels using the pseudo-label filtering algorithm by relying on the video-level ground-truth annotation and the pseudo-label confidence scores.

FIG. 6B is a block diagram illustrating a workflow 650 for training a model using unlabeled data, according to principles of the present disclosure. When trained using the workflow 650, the model can be applied to generate predictions based on medical imaging data, such as frame-level predictions and/or video-level predictions. The workflow 650 can be performed, for example, by the cineloop localization processor 210 of FIG. 2. Additionally or alternatively, the model trained using the workflow 650 can be included in the AI model 142 of FIG. 1.

To train the model, an input 655 is received comprising medical imaging data, such as ultrasound cineloops comprising a plurality of frames. The received medical imaging data includes at least some unlabeled data. The workflow 650 can include applying teacher-student training using the unlabeled data, such as by pre-training a teacher model 660, as described in Stage 1 of the process 500 of FIG. 5, and applying mutual learning using the pre-trained teacher model 660 and a student model 665, as described in Stage 2 of the process 500 of FIG. 5. For example, the teacher model 660 can be pre-trained using a portion of the input 655 that includes frame-level annotations, and the teacher model 660 and the student model 665 can be jointly trained using a portion of the input 655 that includes unlabeled data. The teacher model 660 and the student model 665 are both trained to generate frame-level predictions (e.g., 670), and the frame-level annotations generated by the teacher model 660 are used as pseudo-labels 675 to serve as a ground truth for training the student model 665.

To enable video-level supervision, per-frame predictions are aggregated into one or more video-level predictions 680. This can be achieved using one or more frame-to-video feature encoders 685, which combine predictions from a plurality of frames into a video-level prediction 680. Video-level predictions 680 generated using the student model 665 can be compared against video-level pseudo-labels 690 generated by the teacher model 660 to determine a loss, L_{cls_v} for the video-level predictions. The loss L_{cls_v} is back propagated to improve accuracy of video-level predictions.

The one or more frame-to-video feature encoders 685 process frame-level predictions from the baseline detection/segmentation network and output a video-level classification prediction for the video input. A video-level prediction 680 can then be compared against a video-level pseudo-label 690 for that clip. The video-level loss is computed as a function of the difference between the prediction 680 and the pseudo-label 690; this occurs during the mutual learning stage of the teacher-student training. The video-level classification loss can be a binary cross-entropy loss. During training, this loss is back propagated through the student network to update the student model's 665 weights.

In some implementations, the workflow 650 optionally includes applying a filtering component 695 to improve accuracy of pseudo-labels 675 generated by the teacher model 660. The effectiveness of the mutual learning training process may be at least partially dependent on the quality of pseudo-labels 675 generated by the teacher model 660, since the pseudo-labels 675 serve as the ground-truth for the student model 665. In some implementations, lower quality pseudo-labels 675 can be filtered away using a filtering algorithm, so that the student model 665 is trained only using filtered pseudo-labels 696, which have been filtered using the filtering component 695 to include only generated pseudo-labels 675 determined to have a quality beyond a threshold level.

To evaluate quality of pseudo-labels 675, a filtering algorithm of the filtering component 695 receives an input comprising a batch of frame-level pseudo-labels 675 predicted by the teacher model 660. The filtering algorithm compares each frame-level pseudo-label 675 to a corresponding video-level pseudo-label (e.g., 690). If the video-level pseudo-label is negative (suggesting that the target feature is not present in the video), all frame-level pseudo-labels 675 for that feature are removed. On the other hand, if the video-level pseudo-label is positive for the target feature, the algorithm checks whether there is at least one frame-level pseudo-label present (above a pre-defined threshold t1). In the case where there is at least one frame-level pseudo-label present, the algorithm removes the frame-level pseudo labels 675 if their maximum confidence scores are below another pre-defined threshold t2. If none of the maximum confidence scores of the predicted frame-level pseudo-labels 675 are above the pre-defined threshold t1, the predicted frame-level pseudo-label 675 that has the maximum confidence score is kept and used as the final predicted frame-level pseudo-label 675 for the entire video clip. Based on the foregoing filtering operations, the filtering component 695 outputs filtered pseudo-labels 696, which can be used to train the student model 665 and/or provided to the one or more frame-to-video feature encoders 685 to generate video-level predictions 680 and/or video-level pseudo-labels 690. Other variations may be applied to evaluate quality of pseudo-labels using the pseudo-label filtering algorithm by relying on the video-level ground-truth annotation and the pseudo-label confidence scores.

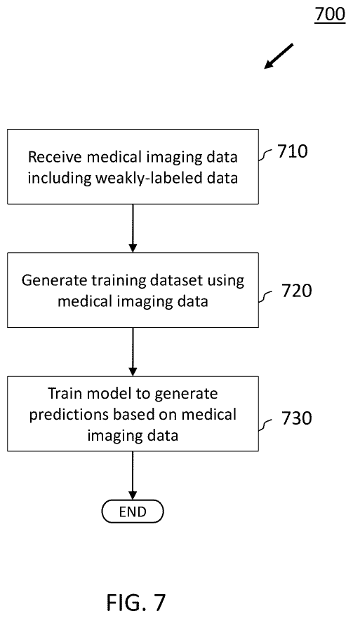

FIG. 7 is a flow diagram illustrating a process 700 for training a model using weakly-labeled data, according to principles of the present disclosure. The process 700 can be performed, for example, using the processor 137 of FIG. 1 to train one or more models included in the AI model 142. When trained using the process 700, the model can be applied to perform feature localization (e.g., detection or segmentation) based on received medical imaging data. The model trained using the process 700 can include for example, a baseline model, a teacher model, and/or a student model, and the process 700 can be used in conjunction with the any of workflows 200, 300, 400, 600, or 650, and process 500 to train one or more models to generate predictions using weakly-labeled data.

The process 700 begins at block 710, where a plurality of medical imaging data is received. The plurality of medical imaging data includes a first set of medical imaging data comprising frame-level annotations and a second set of medical imaging data comprising weakly-labeled data. As described herein, the weakly-labeled data can include unlabeled data and/or data containing only video-level annotations. The medical imaging data can comprise, for example, ultrasound cineloops, each cineloop including a plurality of frames. The frame-level annotations can include indications of target features in frames, such as bounding boxes or segmentation masks. The video-level annotations can include indications of target features in videos, such as binary indications of presence or absence of a target feature or category information for the video.

The process 700 proceeds to block 720, a training dataset is generating using the plurality of medical imaging data received at block 710. The training dataset can comprise frame-level ground truth data. In implementations where the model includes a teacher model and a student model, generating the training dataset can include pre-training the teacher model using the first set of medical imaging data to generate frame-level annotations, using the pre-trained teacher model to generate pseudo-labels based on the weakly-labeled data, and using the generated pseudo-labels to train the student model.

The process 700 proceeds to block 730, where the model is trained to generate predictions based on new medical imaging data. For example, the model can be trained to detect a target feature in medical imaging data (e.g., ultrasound cineloops) and/or the model can be trained to perform segmentation on medical imaging data. In some implementations, the model can be trained to generate video-level annotations, such as annotations indicating a category for the new medical imaging data selected from two or more categories. The new medical imaging data can comprise ultrasound video, which can be recorded or captured in real time. In an example, new medical imaging data is medical imaging data obtained after the generation of the training dataset. In some implementations, training the model can include applying pseudo-label filtering to pseudo-labels generated by the teacher model. In some implementations, training the model can include applying a frame-to-video feature encoder to generate video-level annotations. In some implementations, training the model can include adjusting weights of a teacher model based on weights of a student model, and in some implementations, the adjustments can be applied using adaptive learning.

In some implementations, the process 700 includes applying the trained model to generate predictions using the new medical imaging data. The trained model receives the new medical imaging data and processes the medical imaging data to generate frame-level predictions and/or video-level predictions. For example, the frame-level predictions can include bounding boxes indicating locations of predicted target features or delineations of boundaries of predicted target features. Video-level predictions can include predicted categories for a video, such as predictions as to whether a target feature is present or absent in the video.

In some implementations, process 700 includes testing to evaluate accuracy of the trained model and/or validate the trained model. For example, a portion of the medical imaging data (e.g., 10%) received at block 710 can be excluded from the training dataset and used as test data to evaluate the accuracy of the trained model. The trained model is applied to the test data to determine whether the model accurately performs feature localization (e.g., by comparing outputs of the trained model to ground truth data) with an accuracy beyond a threshold level (e.g., 70% accurate, 80% accurate, 90% accurate, etc.). If the trained model does not exceed the threshold accuracy when applied to the test data then the model can be retrained or discarded in favor of a more accurate model. Retraining the model can include training the model at least a second time using the same training dataset, training the model with a different (e.g., expanded) training dataset, applying different weights to a training dataset, rebalancing a training dataset, and so forth.

The systems, methods, and apparatuses disclosed herein may provide techniques to train one or more segmentation models and/or detection models to perform feature localization using medical imaging data, such as ultrasound imaging data. The disclosed techniques may include applying one or more trained models to perform feature localization. The techniques disclosed herein may reduce or eliminate the need for time-consuming and expensive frame-by-frame annotation efforts.

In various examples where components, systems and/or methods are implemented using a programmable device, such as a computer-based system or programmable logic, it should be appreciated that the above-described systems and methods can be implemented using any of various known or later developed programming languages, such as “Python”, “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software, and/or firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instructions to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, examples or processes described herein may be combined with one or more other examples, examples and/or processes or be separated and/or performed amongst separate devices or device portions in accordance with the present systems, devices and methods.

Finally, the above-discussion is intended to be merely illustrative of the present systems and methods and should not be construed as limiting the appended claims to any particular example or group of examples. Thus, while the present system has been described in particular detail with reference to exemplary examples, it should also be appreciated that numerous modifications and alternative examples may be devised by those having ordinary skill in the art without departing from the broader and intended spirit and scope of the present systems and methods as set forth in the claims that follow. Accordingly, the specification and drawings are to be regarded in an illustrative manner and are not intended to limit the scope of the appended claims.

What is claimed is:

1. A method of training a model to generate predictions using medical images, the method comprising:
receiving a plurality of medical imaging data wherein the plurality of medical imaging data includes a first set of medical imaging data comprising frame-level annotations and a second set of medical imaging data comprising weakly-labeled data;
generating a training dataset, wherein the training dataset comprises frame-level ground truth data; and
training, using the generated training dataset, a model to generate predictions based on new medical imaging data, wherein the generated predictions include frame-level feature localizations.

2. The method of claim 1, wherein the weakly-labeled data comprises at least one of unlabeled data or video-level labeled data.

3. The method of claim 1, wherein the generated predictions include video-level annotations.

4. The method of claim 3, wherein the model is trained to determine a category for the new medical imaging data, and wherein the category is selected from at least two categories.

5. The method of claim 3, wherein the video-level annotations are generated using a frame-to-video feature encoder.

6. The method of claim 1, wherein the new medical imaging data comprises an ultrasound video loop, and wherein the plurality of medical imaging data comprises ultrasound videos, ultrasound frames, or both.

7. The method of claim 1, wherein the model is trained to generate a bounding box indicating a location of a target feature or delineate the location of the target feature.

8. The method of claim 1:
wherein generating the training dataset includes pre-training a teacher model, using the first set of the medical imaging data comprising the frame-level annotations, to generate pseudo-labels; and
wherein training the model includes jointly training the teacher model and a student model using the second set of the medical imaging data comprising the weakly-labeled data, wherein the generated pseudo-labels are used as a ground truth for training the student model.

9. The method of claim 8, further comprising:
transferring weights from the trained student model to the trained teacher model based on a transferring rate determined using an exponential moving average function.

10. The method of claim 9, wherein the transferring rate is adjusted based on evaluating performance of the student model using validation data.

11. The method of claim 8, wherein a frame included in the weakly-labeled data is weakly augmented for training of the teacher model and the frame is strongly augmented for training of the student model.

12. The method of claim 8, further comprising:
evaluating quality of frame-level pseudo-labels included in the generated pseudo-labels based on video-level ground truth annotations or video-level pseudo-labels; and
filtering the frame-level pseudo-labels based on the quality.

13. The method of claim 1, further comprising:
applying the trained model to the new medical imaging data to generate the predictions.

14. The method of claim 1, further comprising:
evaluating an accuracy of the trained model using a testing dataset; and
retraining the trained model using a different training dataset when the accuracy does not exceed a threshold accuracy.

15. The method of claim 1, wherein the model includes a baseline segmentation model or a baseline detection model.

16. A non-transitory computer-readable medium carrying instructions that, when executed by a processor, cause the processor to perform operations comprising:
receiving a plurality of medical imaging data wherein the plurality of medical imaging data includes a first set of medical imaging data comprising frame-level annotations and a second set of medical imaging data comprising weakly-labeled data;
generating a training dataset, wherein the training dataset comprises frame-level ground truth data; and
training, using the generated training dataset, a model to generate predictions based on new medical imaging data, wherein the generated predictions include frame-level feature localizations.

17. The non-transitory computer-readable medium of claim 16:
wherein generating the training dataset includes pre-training a teacher model, using the first set of the medical imaging data comprising the frame-level annotations, to generate pseudo-labels; and
wherein training the model includes jointly training the teacher model and a student model using the second set of the medical imaging data comprising the weakly-labeled data, wherein the generated pseudo-labels are used as a ground truth for training the student model.

18. The non-transitory computer-readable medium of claim 17, wherein the operations further comprise:
transferring weights from the trained student model to the trained teacher model based on a transferring rate determined using an exponential moving average function.

19. The non-transitory computer-readable medium of claim 18, wherein the transferring rate is adjusted based on evaluating performance of the student model using validation data.

20. The non-transitory computer-readable medium of claim 17, wherein the operations further comprise:
evaluating quality of frame-level pseudo-labels included in the generated pseudo-labels based on video-level ground truth annotations or video-level pseudo-labels; and
filtering the frame-level pseudo-labels based on the quality.
